(19) United States
(12) Patent Application Publication
(10) Pub. No.: US 2003/0109432 A1
(43) Pub. Date: Jun. 12, 2003

(54) Anticancer polypeptide-metal complexes and compositions, methods of making, and methods of using same

(76) Inventors: William W. Zuo, Sugar Land, TX (US); Dongfang Yu, Houston, TX (US); David J. Yang, Sugar Land, TX (US); Jing Ya Xu, Missouri City, TX (US)

Correspondence Address: J. M. (Mark) Gilbreth, GILBRETH & ASSOCIATES, P.C., P.O. Box 2428, Bellaire, TX, 77402-2428, US

(21) Appl. No.: 09/940,180

(22) Filed: Dec. 10, 2001

(51) Int. Cl. A61K 38/16; C07K 14/00

(52) U.S. Cl. 514/12; 530/350; 530/410; 530/409

(57) ABSTRACT

Novel drug complexes comprising a polypeptide carrier moiety comprising glutamic acid and at least one of the group consisting of aspartic acid, alanine, asparagine, glutamine, glycine, and any combinations thereof, are disclosed. The drug moiety is a therapeutic metal selected from the group consisting of platinum, iron, gadolinium, rhenium, manganese, cobalt, indium, gallium or rhodium. Methods for making said complexes, compositions comprising said complexes, methods for making saiduch compositions, and methods for treating a patient comprising use of said complexes and/or compositions are further disclosed.

### BACKGROUND OF THE INVENTION

1. Field of the Invention

The present invention relates to polypeptide-transition-metal complexes. In another aspect, the present invention relates to methods for making such polypeptide-transition metal complexes. In even another aspect, the present invention relates to compositions comprising polypeptide- transition-metal complexes and method of making such compositions. In still another aspect, the present invention relates to methods of using such polypeptide-transition-metal complexes and compositions comprising such complexes to treat a patient afflicted with a condition, such as for example a cancer in any stage of development.

2. Description of the Related Art

Improvement of cancer treatment is extensively determined by the development of more tumor specific pharmaceuticals and new drug delivery techniques. Due to an angiogenesis process involved in the tumor vasculature density and permeability, cell cycle regulation and cell signalling agents have opened a new era in the treatment of various tumors and undergone extensive development and evaluation. Despite the outstanding advances made in the field of pharmacology, some significant limitations still remain in the treatment of various diseases via drug agents. One of the most significant limitations at this time relates to the delivery of particular drugs in vivo, especially in situations where drugs are poorly water soluble. Indeed, the use of some drugs which show great promise in vitro, has been severely limited due to issues related to their solubility. This causes problems with drug delivery in vivo. One example of such a drug is cisplatin in the treatment of solid tumors.

As discussed below, the prior art has attempted to address this issue in a number of ways. However, as presented in more detail below, prior to the instant invention, the unique advantages of conjugating a transition-metal drug to one of the inventive polypeptides, while desired, were unknown.

U.S. Pat. No. 4,675,381, issued to Bichon, on Jun. 23, 1987, entitled “Biodegradable Polypeptide and its Use for the Gradual Release of Drugs,” discloses a polyaspartate and/or polyglutamate polymer as a drug carrier. This patent envisions the use of polyaspartate and/or polyglutamate polymers as drug carriers wherein the drug is encapsulated or incorporated in the matrix of the polymer. The patent does not disclose, teach or suggest metal complexes with the polymer. Furthermore, most of the teaching in the patent is directed to homopolymers of aspartate or glutamate, not combinations of the two amino acids.

U.S. Pat. No. 5,087,616 issued to Myers et al. on Feb. 11, 1992, entitled “Cytotoxic Drug Conjugates and Their Delivery to Tumor Cells,” discloses the use of a biodegradable polymeric carrier to which one or more cytotoxic molecules, for instance, daunomycin is conjugated. The biodegradable polymeric carrier is specified to be, for example, a homopolymer of polyglutamic acid. However, the use of a metal complexed with a polypeptide carrier comprising glutamic acid and at least one of the group consisting of aspartic acid, alanine, asparagine, glutamine, glycine, and any combinations thereof, is not disclosed, taught or suggested in this reference.

A 1983 J Med Chem. paper by Piper et al. entitled “A Synthetic Approach to Poly (γ-glutamyl) Conjugates of Methotrexate” discloses the use of methotrexate conjugated to 2 to 3 glutamic acid units. This paper does not disclose, teach or suggest a metal complex to a polypeptide carrier comprising glutamic acid and at least one of the group consisting of aspartic acid, alanine, asparagine, glutamine, glycine, and any combinations thereof.

A 1982 Int. J. Cancer paper by Zunino et al. entitled “Anti-Tumor Activity of Daunorubicin Linked to Poly-L-Aspartic Acid” discloses daunorubicin bound to a homopolymer of polyaspartic acid. The paper indicates that “the binding (of daunorubicin) to the polypeptide markedly reduced drug toxicity but only slightly decreased drug potency.” “The daunorubicin-poly-L-aspartic acid conjugate demonstrated anti-tumor activity comparable to that of doxorubicin in leukemia models, but superior to that of doxorubicin in a solid tumor model.” While this paper does disclose the covalent conjugation of an anti-tumor drug to a homopolymer of polyaspartic acid, it does not disclose, teach or suggest the use of a metal complex to a polypeptide carrier comprising glutamic acid and at least one of the group consisting of aspartic acid, alanine, asparagine, glutamine, glycine, and any combinations thereof.

A 1998 Cancer Research paper by Li et al. entitled “Complete Regression of Well-established Tumors Using a Novel Water-soluble Poly (L-Glutamic Acid)-Paclitaxel Conjugate,” discloses the use of a water-soluble poly-L-glutamic acid-paclitaxel conjugate to produce tumor effects with diminished toxicity. However, this paper does not disclose, teach or suggest the use of the metal-polypeptide carrier complex of the present invention.

A 1989 J. Pharm. Exp. Ther. paper by Ramsammy entitled “Polyaspartic Acid Protects Against Gentamicin Nephrotoxicity in the Rat,” discloses the use of poly-amino acids, including polyaspartic acid, to provide protection against the development of amino glycoside-induced nephrotoxicity in the rat. However, this paper does not disclose, teach or suggest the inventive a metal complex to a polypeptide carrier comprising glutamic acid and at least one of the group consisting of aspartic acid, alanine, asparagine, glutamine, glycine, and any combinations thereof

A 1990 Biopolymers paper by Hayashi and Iwatsuki, entitled “Biodegradation of Copoly (L-aspartic Acid/L-Glutamic Acid) In Vitro,” discloses the preparation of copolypeptides consisting of L-aspartic acid and L-glutamic acid. The paper describes the use of such polypeptides to determine the effects of copolymer composition and sequential distributions on the rate of degradation by papain to stimulate in vivo polymer degradation. This paper does disclose, teach or suggest the use of copolymers of glutamic acid and aspartic acid, similar to the copolymer of the present invention. The paper also does not disclose, teach or suggest the use of a metal complex to a polypeptide carrier comprising glutamic acid and at least one of the group consisting of aspartic acid, alanine, asparagine, glutamine, glycine, and any combinations thereof.

U.S. Pat. No. 4,960,790 issued to Stella et al., and entitled “Derivatives of Taxol, Pharmaceutical Compositions Thereof and Methods for the Preparation Thereof” discloses the anti-tumor agent taxol covalently conjugated with, for example, an amino acid (for example, glutamic acid). However, this patent does not disclose, teach or suggest the use of a metal complex to a polypeptide carrier comprising glutamic acid and at least one of the group consisting of aspartic acid, alanine, asparagine, glutamine, glycine, and any combinations thereof.

Finally, a 1960 J. Am. Chem. Soc. by Karlson et al., entitled “The Helical Sense of Poly-β-benzyl-L-aspartate” discusses the physical characteristics of series of copolymers derived from γ-benzyl-L-glutamate and β-benzyl-L-aspartate. However this paper does not disclose, teach or suggest the use of a metal complex to a polypeptide carrier comprising glutamic acid and at least one of the group consisting of aspartic acid, alanine, asparagine, glutamine, glycine, and any combinations thereof.

As indicated from the above art, there exists a long-felt need in the art to solubilize poorly soluble drugs, such as anti-tumor agents. Thus, there is a need in the art for therapeutic compounds comprising a transition metal drug wherein the compounds have improved solubility in comparison to conventional transition metal drugs.

There is another need in the art for methods of making such therapeutic compounds.

There is even another need in the art for compositions comprising such therapeutic compounds.

There is still another need in the art for methods of making such compositions.

There is yet another need in the art for methods for treating a patient afflicted with a condition such as a cancer in any stage of development.

### SUMMARY OF THE INVENTION

It is an object of the present invention to provide therapeutic compounds comprising a transition metal drug wherein the compounds have improved solubility in comparison to conventional transition metal drugs.

It is another object of the present invention to provide methods of making such therapeutic compounds.

It is even another object of the present invention to provide for compositions comprising such therapeutic compounds.

It is still another object of the present invention to provide methods of making such compositions.

It is yet another object of the present invention to provide methods for treating a patient afflicted with a condition such as a cancer in any stage of development.

According to one embodiment of the present invention there is provided a therapeutic compound comprising at least one therapeutic metal, and at least one polypeptide carrier moiety, the metal complex to the carrier moiety, and the polypeptide drug carrier moiety comprising glutamic acid and a second amino acid selected from the group consisting of aspartic acid, alanine, asparagine, glutamine, glycine, and combinations of two or more amino acids selected from the group consisting of aspartic acid, alanine, asparagine, glutamine, and glycine. A preferred second amino acid is aspartic acid.

Generally the polypeptide drug carrier moiety comprises from about 50 to about 90 percent, by total weight of the carrier, glutamic acid, and from about 10 to about 50 percent, by total weight of the carrier, aspartic acid, or alanine, or asparagine, or glutamine, or glycine, or combinations thereof; more preferably from about 60 to about 80 percent, by total weight of the carrier, glutamic acid, and from about 20 to about 40 percent, by total weight of the carrier, aspartic acid, or alanine, or asparagine, or glutamine, or glycine, or combinations thereof; and most preferably from about 70 to about 75 percent, by weight, glutamic acid, and from about 25 to about 30 percent, by weight, aspartic acid, or alanine, or asparagine, or glutamine, or glycine, or combinations thereof.

Generally the drug moiety is selected from the group consisting of therapeutic metals. Preferably, the therapeutic metals are platinum, iron, gadolinium, rhenium, manganese, cobolt, indium, gallium or rhodium. A preferred drug moiety is platinum.

Generally the drug moiety of the therapeutic metal comprises from about 10 percent to about 60 percent, by weight, more preferably from about 20 percent to about 50 percent, by weight, and most preferably from about 20 percent to about 40 percent, by weight of the therapeutic compound. Moreover, the polypeptide carrier moiety may comprise from about 40 percent to about 90 percent, by weight, more preferably from about 50 percent to about 80 percent, by weight, and most preferably from about 60 percent to about 80 percent, by weight of the therapeutic anticancer peptide-metal complex.

According to another embodiment of the present invention there is provided methods for making a therapeutic compound. Generally

According to even another embodiment of the present invention there is provided a composition comprising a therapeutic compound.

According to still another embodiment of the present invention there is provided a method for making a composition comprising a therapeutic compound.

According to yet another embodiment of the present invention there is provided a method for treating cancer comprising the steps of administering a therapeutically effective amount of an anticancer peptide comprising at least one metal, and at least one polypeptide drug carrier moiety, the metal being covalently chelated to the carrier moiety, and the polypeptide drug carrier moiety comprising glutamic acid and a second amino acid selected from the group consisting of aspartic acid, alanine, asparagine, glutamine, glycine, and combinations of aspartic acid, alanine, asparagine, glutamine, and glycine.

These and other embodiments of the present invention will become apparent to those of skill in the art upon review of this specification, including its drawings, appendix, and claims.

### BRIEF DESCRIPTION OF THE DRAWINGS

FIG. 1A is a schematic illustrating the synthesis of a poly(glutamidaspartic acid)polypeptide.

FIG. 1B provides results from an amino acid analysis of a sample of the poly(glutamic/aspartic acid polypeptide.

FIG. 1C shows an NMR spectra of a sample of poly(glutamate/aspartame).

FIG. 2 is a synthetic scheme illustrating platinum (II)- and (IV)-poly(glutamate/aspartame)complexes.

FIG. 3 shows elemental analysis of the platinum-poly(glutamate/aspartate)complexes Pt(II) (PDDP), Pt (IV) (PPAP) , and cis-1,2-DACH—Pt SO₄(DACH).

FIGS. 4A-C shows results from in vitro cell culture assays of cisplatin (CDDP) and poly(glutamate/aspartate)acid-1,2-DACH—Pt (II) complex (PDDP) in sarcoma (4A) and prostate cancer cell lines (4B and 4C).

FIG. 5 shows the in vivo antitumor activity of an inventive poly(glutamate/aspartate)-1,2-DACH—Pt (II) complex compared to saline (control) in rats bearing breast tumors.

FIG. 6 shows specific cellular target expression changes at 48 hours post treatment of cells with poly(glutamate/aspartate)-1,2-DACH—Pt (II) complex (PDDP), cisplatin (CDDP), and saline (Control).

FIG. 7 shows histopathological changes at 48 hours post treatment of poly(glutamate/aspartate)-1,2-DACH—Pt (II) complex (PDDP) , cisplatin (CDDP) , and saline (Control) . A marked necrosis and apoptosis were noted post-treatment.

### DETAILED DESCRIPTION OF THE INVENTION

The present invention relates to the discovery that polypeptides composed of glutamate and at least of the group consisting of aspartate, alanine, asparagine, glutamine, glycine, and any combinations thereof, make an unexpectedly good carrier for delivery of therapeutic metals, including poorly soluble drugs. An illustrative example includes a polypeptide-platinum complex. The inventive complex of the present invention comprising a metal conjugated to a polypeptide to increase metal solubility in vivo, while desired in the art, has not been anticipated or suggested by the art.

The polypeptide-metal complexes of the invention have improved water solubility, reduced side effects, and are more effective against tumors in comparison to conventional metal drugs. Preferably, the water solubility of the therapeutic anticancer peptide-metal complex is greater than the water solubility of the metal alone. Generally the polypeptide comprises glutamic acid with at least one of the group consisting of alanine, aspartic acid, glycine, glutamine, asparagine, and any combinations thereof. Generally, the drug moiety is a therapeutic metal and may be selected from the group consisting of platinum, iron, gadolinium, rhenium, manganese, cobolt, indium, gallium and rhodium. In a preferred embodiment, the drug moiety is platinum.

As described in more detail below, the present inventors have discovered that the use of a polypeptide drug carrier comprising glutamate and at least one of the group consisting of aspartate, alanine, asparagine, glutamine, glycine, and any combinations thereof, results in unexpectedly good in vivo properties when complexed with metals. Preferred polypeptides of the invention include poly-glutamate/aspartate and poly-glutamate/alanine, asparagine, glutamine, glycine. In particular, and for example, one use of the instant invention involves the complex of platinum to a polypeptide of the invention to enable the effective in vivo treatment of cancer.

Regarding the role of alanine, asparagine, glutamine, and/or glycine in the present invention, the following is noted. It is believed at the time of the application that a polypeptide comprising glutamate and aspartic acid is a preferred polypeptide of the invention However, it is also believed, but not in any limiting sense, that any amino acids similar to aspartic acid, including alanine, asparagine, glutamine, and glycine, can be substituted for aspartic acid in the inventive polypeptide. While not wishing to be bound in anyway, it is believed that a key aspect of the inventive polypeptide relates to the glutamic acid backbone. It is believed that as long as glutamic acid is present in the polypeptide, aspartic acid may serve as the other amino acid, or any amino acid similar to aspartic acid, such as, for example, alanine, asparagine, glutamine, glycine, and any combinations thereof may be used. These amino acids may be substituted in whole or in part for aspartic acid and may be a combination of at least any two of these amino acids. Thus, the present invention provides a plurality of inventive polypeptide polymers, each having glutamic acid, and at least one of the group consisting of aspartic acid, alanine, asparagine, glutamine, glycine, and any combinations thereof.

One embodiment of the present invention relates to a therapeutic compound comprising at least one drug moiety, and at least one polypeptide drug carrier moiety, the drug moiety being complexed to the carrier moiety, and the polypeptide drug carrier moiety comprising glutamic acid and a second amino acid selected from the group consisting of aspartic acid, alanine, asparagine, glutamine, glycine, and combinations thereof. Generally the drug moiety is selected from the group consisting of therapeutic metals. Preferably, the therapeutic metals are platinum, iron, gadolinium, rhenium, manganese, cobolt, indium, gallium or rhodium. A preferred drug moiety is a platinum analogue.

Conditions to be treated may include, but are in no way limited to, prostate, breast, ovarian, colon, leukemia, lymphoma, sarcoma, head and neck, lung and liver cancers, and any combinations thereof. The condition may be in any stage of development.

Based on the total weight of the polypeptide drug carrier moiety, generally the polypeptide drug carrier moiety comprises glutamic acid in an amount ranging from about 50 to about 90 percent, preferably from about 60 to about 80 percent, and more preferably from about 70 to about 75 percent. Based on the total weight of the polypeptide drug carrier moiety, generally the polypeptide drug carrier moiety comprises at least a second amino acid in an amount ranging from about 10 to about 50 percent, preferably from about 20 to about 40 percent, and more preferably from about 30 to about 25 percent. The at least second amino acid is selected from the group consisting of aspartic acid, alanine, asparagine, glutamine, glycine, and all combinations thereof.

Based on the total weight of the therapeutic compound (i.e, the therapeutic peptide-metal complex), the therapeutic compound generally comprises a metal moiety in an amount ranging from about 10 percent to about 60 percent, more preferably from about 20 percent to about 50 percent, and most preferably from about 20 percent to about 40 percent. Based on the total weight of the therapeutic compound (i.e, the therapeutic peptide-metal complex), the therapeutic compound generally comprises the polypeptide drug carrier moiety in an amount ranging from about 40 percent to about 90 percent, more preferably from about 50 percent to about 80 percent, and most preferably from about 60 percent to about 80 percent.

In preferred embodiments, for example, platinum with a polypeptide glutamic acid/aspartic acid carrier), the metal moiety does not comprise more than about 60% by weight of the therapeutic anticancer peptide-metal complex (in order to not adversely affect solubility and/or viscosity which can effect injectability of the compound).

In a particularly preferred embodiment, therapeutic polypeptide-drug complex/compound comprises a molecular weight of about 20,000 to about 50,000 dalton, a platinum drug moiety in an amount ranging from about 20 to about 40 percent based on the total weight of the compound, and a carrier moiety comprising about 70 percent glutamic acid based on the total weight of the carrier moiety, and about 30 percent aspartic acid based on the total weight of the carrier moiety.

Another embodiment of the present invention is directed to a method for making a therapeutic compound. Generally the method comprising the steps of covalently conjugating at least one drug moiety with at least one polypeptide drug carrier moiety to create a therapeutic polypeptide-metal complex of the invention. Generally based on the total weight of the carrier moiety, the carrier moiety comprises from about 50% to about 90% glutamic acid, and from about 10% to about 50% of at least a second amino acid selected from the group consisting of aspartic acid, alanine, asparagine, glutamine, glycine, and any combinations thereof. Preferably the second amino acid is aspartic acid. Generally the drug carrier moiety has a molecular weight from about 20,000 daltons to about 50,000 dalton, and the drug moiety is a therapeutic metal selected from the group consisting of platinum, iron, gadolinium, rhenium, manganese, cobolt, indium, gallium or rhodium. Preferably the drug moiety is platinum, such as 1,2-diaminocyclohexane platinum (II) and 1,2-diaminocyclohexane-dichloro platinum (IV). In particularly preferred therapeutic compound of the invention the carrier moiety comprises about 70 percent glutamic acid and about 30 percent aspartic acid based on the total weight of the carrier moiety, the drug moiety is about 24 percent to about 30 percent by weight of the total weight of the therapeutic compound, the drug moiety is platinum (II) and platinum (IV), and the molecular weight of the therapeutic compound is from about 26,000 to about 30,000 dalton.

Even another embodiment of the present invention is directed to a composition comprising a therapeutic compound. The therapeutic compound may be any of the polypeptide-metal complexes of the present invention. Suitable compositions of the invention are described in detail in the Dosage and Formulation section of the present application.

Still another embodiment of the present invention is directed to a method for making a composition. Generally the method comprises the steps of combining a pharmaceutical carrier with a therapeutic compound of the invention. Suitable carriers are described in detail in the Dosage and Formulation section of the present application.

Yet another embodiment of the present invention is directed to a method for treating a patient afflicted with a condition. Generally the method comprises the step of administering a therapeutically effective amount of a therapeutic compound of the present invention to a patient. Treatment methods, modes of administration and dosages are described in detail in the Dosage and Formulation section of the present application.

Even still another aspect of the present invention relates to a method for improving the solubility of a drug moiety comprising the metal. In a preferred embodiment, the water solubility of the therapeutic compound is greater than the water solubility of the metal alone. In a preferred embodiment, the drug moiety may be platinum analogue.

As used herein, the term “therapeutic”, for example, in the phrases “therapeutic compound” and “therapeutically effective amount” means to have at least some minimal physiological effect. For example, a “therapeutic compound” would have at least some minimal physiological effect upon being administered to a living body/patient. An agent may have at least some minimal physiological effect upon administration to a living body if, for example, administration results in a change in the physiology of the recipient animal. For example, a physiological effect upon administering a “therapeutic” anti-tumor compound may be the inhibition of tumor growth, or decrease in tumor size, or prevention of reoccurrence of the tumor. Administration of a “therapeutically effective amount” means the amount administered is physiologically significant. An agent is physiologically significant if its presence results in a change in the physiology of a recipient animal. For example, in the treatment of cancer or neoplastic disease, a compound which inhibits the growth of a tumor or decreases the size of the tumor or prevents the reoccurrence of the tumor would be considered therapeutically effective.

The term “anti-tumor drug” as used herein means any therapeutic agent having therapeutic effect against a tumor, neoplastic disease or cancer. The term “metal”as used herein means metal having a therapeutic effect when administered to an animal.

The preferred dosage of the present administration for therapeutic treatment is a therapeutically effective dosage/amount of the administered agent sufficient to generate a response when administered to the patient.

The term “treating”, as used herein, for example in the term “treating a condition”, means at least the administration of a therapeutically effective amount of a therapeutic compound to elicit a therapeutic effect. It does not necessarily imply “curing”, but rather implies that the administration of the present invention to a living body afflicted with a condition results in at least some minimal physiological effect upon the condition. For example, treatment could encompass administering an agent wherein the presence of that agent results in a change in the physiology of the recipient animal.

The terms “peptide”, “polypeptide”, “di-peptide”, “copolymer”, “poly(glutamic acid/aspartic acid)” (and all variations thereupon), and “inventive peptide”, refer to the peptide of the present invention as further defined herein (and comprising, for example, a polypeptide comprising aspartic acid and glutamic acid and/or polypeptides comprising aspartic acid with alanine, asparagine, glutamine and glycine, in any combination).

The term “patient” as used herein refers to the recipient to whom the present invention is administered. The patient may be any organism capable of developing cancer, or afflicted with a cancer wherein the cancer is in any stage of development. Preferably, the patient of the invention is a mammal. In a particularly preferred embodiment, the patient is a human.

Dosage and Formulation

The therapeutic compounds (including compounds, drugs, conjugates and the like, as well as compositions comprising the inventive polypeptide-metal complexes,) of this invention can be formulated and administered to treat a variety of conditions. They can be administered by any conventional means available for use in conjunction with pharmaceuticals, either as individual therapeutic active ingredients or in a composition comprising a combination of therapeutic active ingredients. They can be administered alone, or with a pharmaceutical carrier selected on the basis of the chosen route of administration and standard pharmaceutical practice.

The dosages are determined for the chosen therapeutic use, including the condition to be treated, the therapeutic agent used to treat the condition and the type of animal treated (including considerations as to age, weight, sex and so forth). Such determinations are well within the scope of those skilled in the art and do not involve undue experimentation or exercise of inventive skill.

The dosage administered will be a therapeutically effective amount of active ingredient and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired. Usually a daily dosage (therapeutic effective amount) of active ingredient can be about 1 to 400 milligrams per kilogram of body weight. Ordinarily, 1 to 200, and preferably 1 to 50, milligram per kilogram per day given in dividend doses 2 to 4 times a day or in sustained release form is effective to obtain desired results.

Dosage formulations (compositions) suitable for internal administration contain from about 1.0 to about 500 milligrams of active ingredient per unit. In these pharmaceutical compositions, the active ingredient will ordinarily be present in an amount of about 0.05-95% by weight based on the total weight of the composition.

Administration may be by any means suitable for the condition to be treated and may include, for example, oral administration. Such determination is within the ordinary level of skill of one skilled in the art. For example, oral administration may be accomplished using solid dosage forms such as capsules, tablets and powders, or in liquid dosage forms such as elixirs, syrups, emulsions and suspensions.

The therapeutic compound (agent, composition, or the like) may also be, for example, parenterally administered by injection, rapid infusion, nasopharyngeal absorption of dermoabsorption. The agent may also be admimstered intramuscularly, intravenously, or as a suppository.

Gelatin capsules may contain the therapeutic compound and powdered carriers such as lactose, sucrose, mannitol, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestional tract.

Liquid dosage forms for oral administration can contain coloring and flavoring to increase patient acceptance.

In general, water, a suitable oil, saline, aqueous dextrose (glucose), and related sugar solutions and glycols are suitable carriers for parenteral solutions. Solutions for parenteral administration may contain a water soluble salt of the therapeutic compound (agent and the like), suitable stabilizing agents and, if necessary, buffer substances. Antioxidizing agents such as sodium bisulfate, sodium sulfite or ascorbic acid either alone or combined are suitable stabilizing agents. Also used are citric acid and its salts and sodium EDTA. In addition, parenteral solutions can contain preservatives such as benzalkonium chloride, methyl- or propyl-prarben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyaminoacids, polyvinylpyrrolidone, ethylenevinylacetate, methyl cellulose, caraboxymethyl cellulose or protamine sulfate. The concentration of macromolecules as well as a the methods of incorporation can be adjusted in order to control release. Additionally, the agent can be incorporated into particles of polymeric materials such as polyesters, polyaminoacids, hydrogels, poly (lactic acid) or ethylenevinylacetate copolymers. In addition to being incorporated, these agents can also be used to trap the compound in microcapsules. Useful pharmaceutical dosage forms for administration of the compounds of this invention can be illustrated as follows.

Capsules: Capsules may be prepared by filling standard two-piece hard gelatin capsulates each with from about 50 mg to about 150 mg, preferably about 100 mg of powdered active ingredient, about 150 mg to about 200 mg, preferably about 175 mg of lactose, about 15 mg to about 30 mg, preferably about 24 mg of talc and about 1 mg to about 15 mg, preferably about 6 mg magnesium stearate.

Soft Gelatin Capsules: A mixture of active ingredient in soybean oil may be prepared and injected by means of a positive displacement pump into gelatin to form soft gelatin capsules containing from about 50 to about 150 mg, preferably 100 mg of the active ingredient. The capsules are then washed and dried.

Tablets: Tablets may be prepared by conventional procedures so that the dosage unit is from about 50 mg to about 150 mg, preferably about 100 mg of active ingredient, 0.1 to about 0.5 mg, preferably about 0.2 mg of colloidal silicon dioxide, from about 1.0 mg to about 10 mg, preferably about 5 mg of magnesium stearate, from about 200 to about 300 mg, preferably about 275 mg, of microcrystalline cellulose, about 5 to about 15 mg, preferably about 11 mg of cornstarch and about 75 to about 120 mg, preferably about 98.8 mg of lactose. Appropriate coatings may be applied to increase palatability or to delay absorption.

Injectable: A parenteral composition suitable for administration by injection is prepared by stirring about 1.0 to about 5.0% by weight, preferably about 1.5% by weight, of active ingredients in about 5 to about 20% by volume, preferably about 10% by volume, propylene glycol and water. The solution is made isotonic with sodium chloride and sterilized.

Suspension: An aqueous suspension is prepared for oral administration so that from about 1 to about 10 ml, preferably about 5 ml contain about 50 to about 150 mg, preferably about 100 mg of finely divided active ingredient, about 150 to about 250 mg, preferably about 200 mg of sodium carboxymethyl cellulose, about 2 to about 10 mg, preferably about 5 mg of sodium benzoate, about 0.1 to about 5 g, preferably about 1.0 g of sorbitol solution U.S.P. and from about 0.01 to about 0.1 ml, preferably about 0.025 ml of vanillin.

The amino acids useful in the polypeptides of the present invention may be D amino acids, L amino acids, or mixtures of D and L amino acids. Further, it is contemplated that the carrier polypeptide of the present inventive complexes need not exclusively contain an individual polypeptide containing solely the combination of glutamic and at least one of the group consisting of aspartic acid, alanine, asparagine, glutamine, glycine, and all combinations thereof. Rather, while sections of the polypeptide may contain the noted combination of amino acids, it is believed that it is not necessary for the entire peptide to homogeneously include only the noted amino acids, especially not necessarily in repeating monomers. Thus, the carrier may comprise components other than the noted amino acids, providing that at least some of the carrier polypeptide is composed of the inventive combinations of amino acids.

It is readily apparent to one skilled in the art that various substitutions and modifications may be made to the invention disclosed herein without departing from the scope and spirit of the invention.

All references cited in the present application, including journal articles, laboratory manuals, all U.S. and foreign patents and patent applications, are specifically and entirely incorporated by reference.

### EXAMPLES

The following examples are provided to illustrate the present invention. These examples are not intended to limit the scope of the claims of the present invention, and should not be so interpreted.

In order to demonstrate one embodiment of the present invention, an antitumor transition metal drug, platinum, was made with an inventive polypeptide of the invention (for example, a polypeptide comprising glutamic acid and aspartic acid) and used as a drug delivery vehicle. This inventive conjugate possesses superior biological and therapeutic properties in vivo over, for example, non-polymeric metal complex. Data provided herein shows that, for example, chelating platinum to an inventive polymer of the invention resulted in unexpected therapeutic properties of platinum, such as the treatment of cancer.

Platinum was selected to serve as an exemplary embodiment of a drug to be conjugated with the inventive carrier because platinum is known in the art to be an antitumor drug and known to have solubility problems in vivo. Hence, it has known effectiveness problems and toxicity problems in vivo related to its stability and related in vivo use. Furthermore, the need for the present invention (i.e., a carrier that can solubilize and/or enhance the in vivo therapeutic use of drugs, for example, poorly soluble drugs, such as, for example, poorly soluble antitumor drugs) is amply demonstrated by platinum since as discussed in the background of the present application, there have been numerous attempts in the prior art to conjugate the drug to various carriers, including polypeptides, in attempts to improve the biological applicability of platinum.

The effectiveness of platinum complexes against tumor activity has been demonstrated. For instance, cisplatin, a widely used anticancer drug, has been used alone or in combination with other agents. Cisplatin causes cell arrest at S-phase and that leads to mitotic arrest of proliferating cells. Cisplatin also decreases expression of vascular endothelial growth factor (VEGF) during chemotherapy. Cisplatin is effective in the treatment of majority solid tumors. However, its clinical applications are associated with significant nephrotoxicity, myelosuppression, drug resistance, gastrointestinal toxicity, neurotoxicity and other side effects (e.g. vomiting, granulocytopenia and body weight loss. Cisplatin is formulated in the bulky vehicles and has been used to treat breast, ovarian, colon and lung cancers. Its poor water solubility greatly impairs its therapeutic efficacy. Chemical modification of cisplatin may increase its hydrophilicity, reduce its side effect, and improve its therapeutic efficacy. However, because of the chemical inertness of platin, modification of platin derivatives to be more potent and to have better water solubility than cisplatin has been less demonstrated.

Platinum was, therefore, chosen, as an exemplary drug in which to complex to the inventive glutamic acid/aspartic acid polypeptide in order to determine whether conjugation in the inventive complex produces a drug carrier complex which shows improved therapeutic use.

### Example 1

Synthesis of Polypeptide: Poly(glutamate/aspartate)

N-carboxyanhydride (NCAs) was prepared by phosgenation (a procedure known in the art) of the corresponding β-benzyl-l-aspartate and γ-benzyl-l-glutamate (Idelson, M., Blout, E. R., J. Am. Chem. Soc. 1958, 80, 2387-2393; Karlson, R. H., Norland, K. S., Fasman, G. D., Blout, E. R., J. Am. Chem. Soc. 1960, 82, 2268-2278; Paolillo, L., Temussi, P. A., Bradbury, E. M., Crane-Robinson, C. Biopolymers, 1972, 11, 2043-2052; Hayashi, T., Iwatsuki, M., Biopolymers, 1990, 29, 549-557; Bradbury, E. M., Carpenter, B. G., Crane-Robinson, C., Goldman, H., Macromolecules, 1971, 4, 557-564.).

Briefly, a solution of phosgene (10% w/v) was bubbled into ethylacetate (150 ml). An aliquot (10 ml) of this solution was added to 10 grams of finely ground β-benzyl-l-aspartate and γ-benzyl-l-glutamate in ethylacetate (150 ml). The reaction was stirred under reflux for 5 mm. A stream of nitrogen was employed to remove excess HCI prior to the next addition of phosgene. The sequence was repeated until no traces of suspended amino acid HCI remained. The mixture was then filtered and the solvent was evaporated in vacuo. The product was crystallized from ethyl acetate.

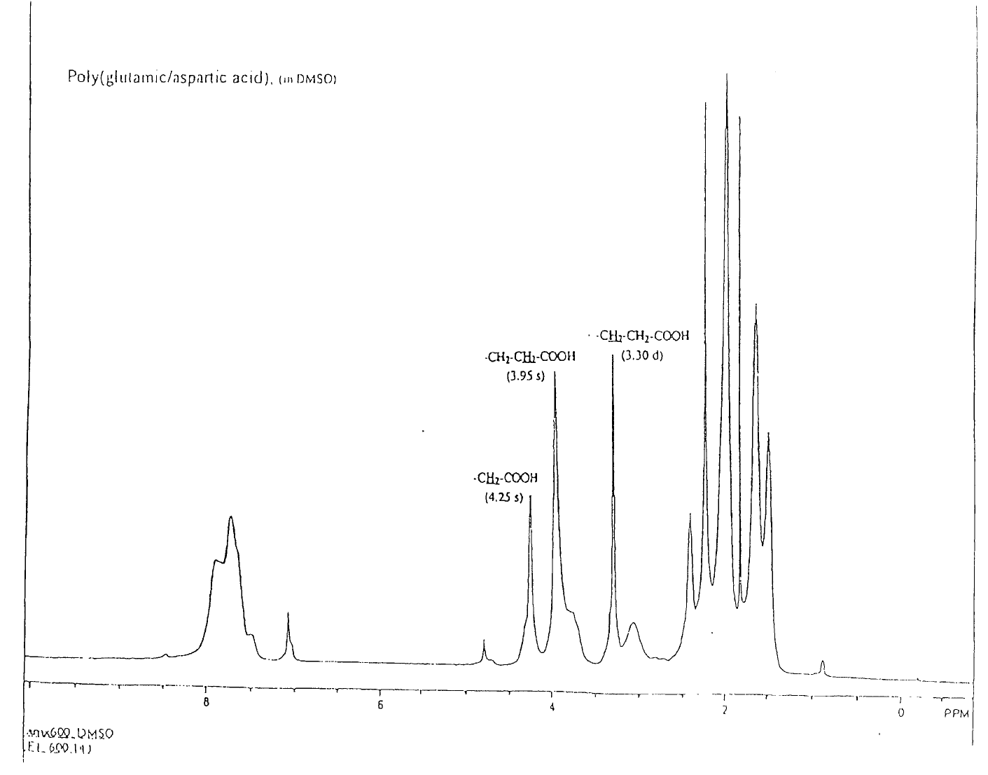

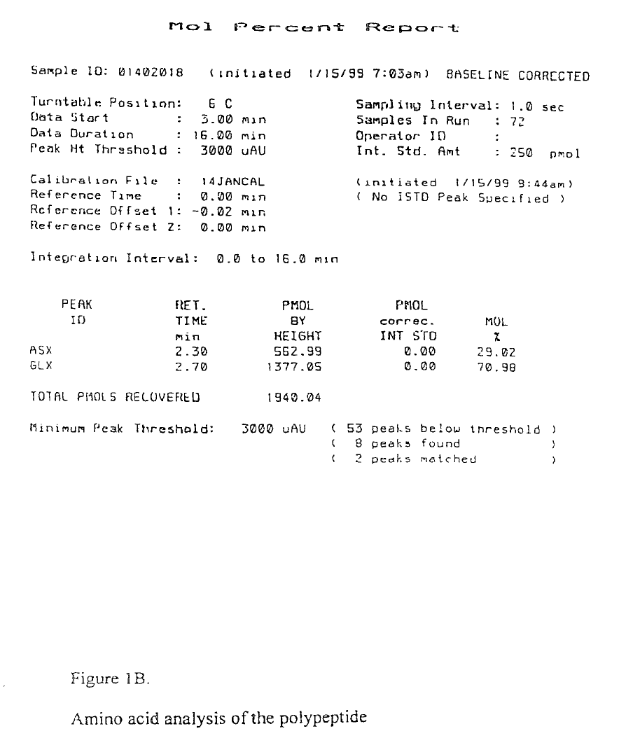

Solutions of NCAs of β-benzyl-l-aspartate and γ-benzyl-l-glutamate in dioxane/methylene chloride (1:3) were prepared. The ratios (w/w) used between δ-benzyl-l-aspartate and γ-benzyl-l-glutamate were 3:7, 2:8 and 1:9. The polymerization was initiated with triethylamine in methylene chloride (4 ml, 2.5% v/v). The copolymerization reaction was under reflux for 30 min and followed by CO₂ evolution. The reaction was stopped at about 30 mol % conversion. The polymers formed were precipitated by adding ice cold methanol containing 0.IN HCI (5%) v/v). The products were washed with methanol and dried under reduced pressure, yielded 8 gm (for 3:7 batch). The debenzylation was conducted by using HBr according to a known procedure (Idelson, M.; Blout, E. R., J. Am. Chem. Soc. 1958, 80, 2387-2393). After HBr treatment, the aqueous solution was dialyzed against distilled water, filtered through Millipore filter and lyophilized. Typical average molecular weight was 26,000-30,000 daltons. A synthetic scheme is shown in FIG. 1A. A similar technique was used to prepare polymers of glutamic acid and alanine, glutamic acid and asparagine, glutamic acid and glutamine, glutamic acid and glycine, and glutamic acid and one or more amino acids from the group consisting of aspartic acid, alanine, asparagine, glutamine, and glycine.

Amino add analyzer (PE/ABI 420 A) (Foster City, Calif.) was used to determine the actual composition ratio of aspartic acid and glutamic acid. Briefly, polypeptide was hydrolyzed with HCI (6N) at 150° C. for 75 min. The hydrolyzed products were loaded on PVDF membrane and methanol (30%) and HCI (0.IN, 0.2 ml) were added to extract the amino acids. Using pre-column derivatization with phenylisothiocyanate, the amino acid concentration was determined. An amino acid analysis of the poly(glutamic acid/aspartic acid) is shown in FIG. 1B. FIG. 1C provides the results of NMR analysis on a sample of the poly(glutamic/aspartic acid) polypeptide.

### Example 2

Synthesis of Polv(qlutamate/aspartate)—Platinum Analoque (II) and (IV) Complex

Numerous studies have suggested that limited polymer-drug conjugate discretion through the kidneys is evident when the molecular weight of the conjugate ranges from 20,000 to 50,000 daltons. Thus, to enhance tumor uptake of the platinum analogue-inventive carrier conjugate, a molecular weight range of a conjugate of 26,000 to 30,000 daltons was selected. It is suggested that Pt (IV) complex exerts its anticancer effect via in vivo reduction to Pt (II) complex. Thus, it is anticipated that Pt (II) is more potent than Pt (IV). Therefore, both Pt (II) and Pt (IV) were synthesized and bound to poly(glutamic/aspartic acid) polypeptide.

Cis-1,2-Diaminocyclohexane sulfatoplatinum (II) (cis-1,2-DACH—Pt×SO₄) was synthesized via a two-step procedure. Cis-1,2-DACH—PtI₂ complex was synthesized by mixing a filtered solution of K₂PtCl₄(5.009, 12 mmol) in 120 ml of deionized water with KI (20.00 g in 12 ml of water, 120 mmol) and was allowed to stir for 5 min. To this solution one equivalent of the cis-1,2-DACH(1.37g, 1.487 ml, 12 mmol) was added. The reaction mixture was stirred for 30 min at room temperature. A yellow solid was obtained which was separated by filtration, washed with a small amount of deionized water. The final product was dried under vacuum, yielded cis-1,2-DACH—PtI₂ (6.48 g, 96%). Without further purification, cis-1,2-DACH—PtI₂ (6.48 g, 11.5 mmol) was added as a solid to an aqueous solution of Ag₂SO₄ (3.45 g, 11 mmol). The reaction mixture was left stirring overnight at room temperature. The AgI was removed by filtration and the filtrate was freeze dried under vacuum, yielded yellow cis-1,2-DACH—Pt(II)SO₄ (4.83 g, 99%). Elemetal analysis Pt: 44.6% (w/w).

Conjugation of platinum (II) analog cis-1,2-DACH—Pt.SO₄ to poly (glutamate/aspartate) was conducted as follows: Cis-1,2-DACH—Pt.SO₄ (500 mg, 1.18 mmol) was dissolved in 10 ml of deionized water, and a solution of sodium poly(glutamic/aspartic acid) (1.00 g; Glu:Asp; 7/3) in 15 ml of deionized water was added. The solution was left stirring for 24 hr at room temperature. After dialysis (MWCO:10,000) and lyophilization, the yield of cis-1,2-DACH—Pt (II)-poly(glutamic/aspartic acid) was 1.1462 g. Elemetal analysis Pt: 17.64% (w/w).

Cis-1,2-DACH-dichloro-Pt (IV)-poly(glutamic/aspartic acid) was synthesized as follows: the above solution was added dropwise 2.5 ml of 30% aqueous hydrogen peroxide. After 24 hr. HCl (75 ml of 0.02 N ) was added and left stirring for 24 hr at room temperature, dialyzed (MWCO:10,000) by deionized water for overnight, freeze dried under vacuum. The final product obtained was 1.15 g. Elemetal analysis Pt: 16.11% (w/w).

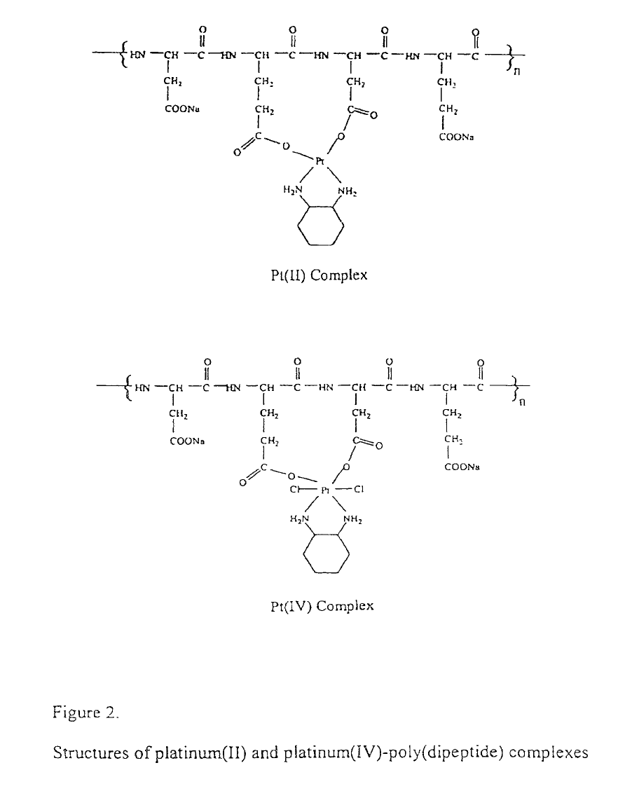

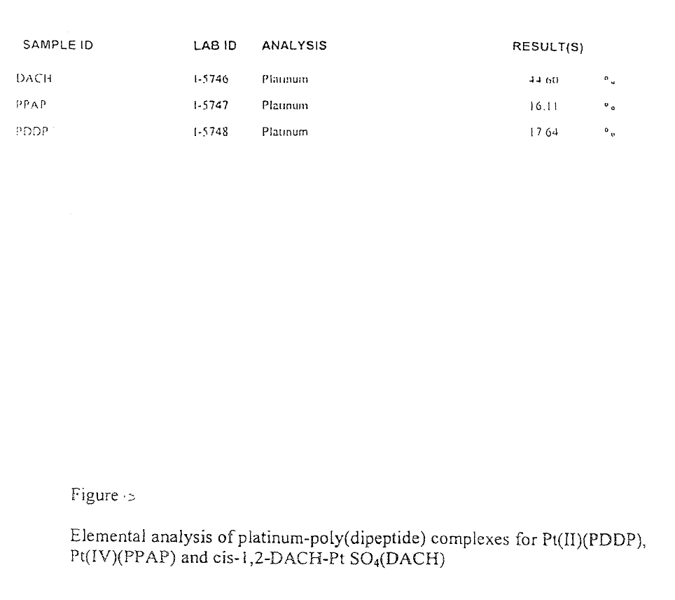

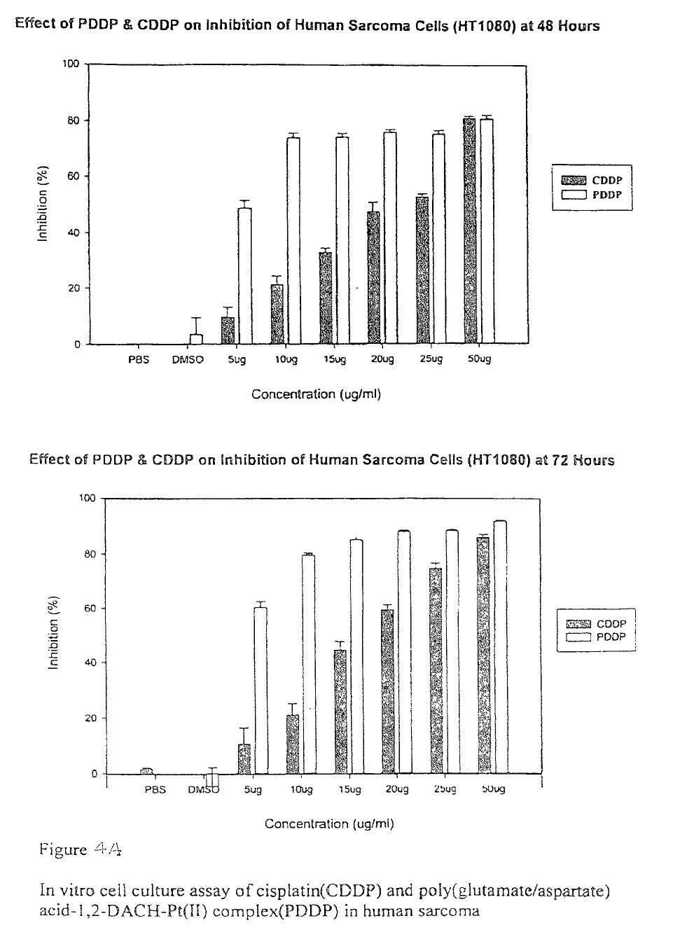

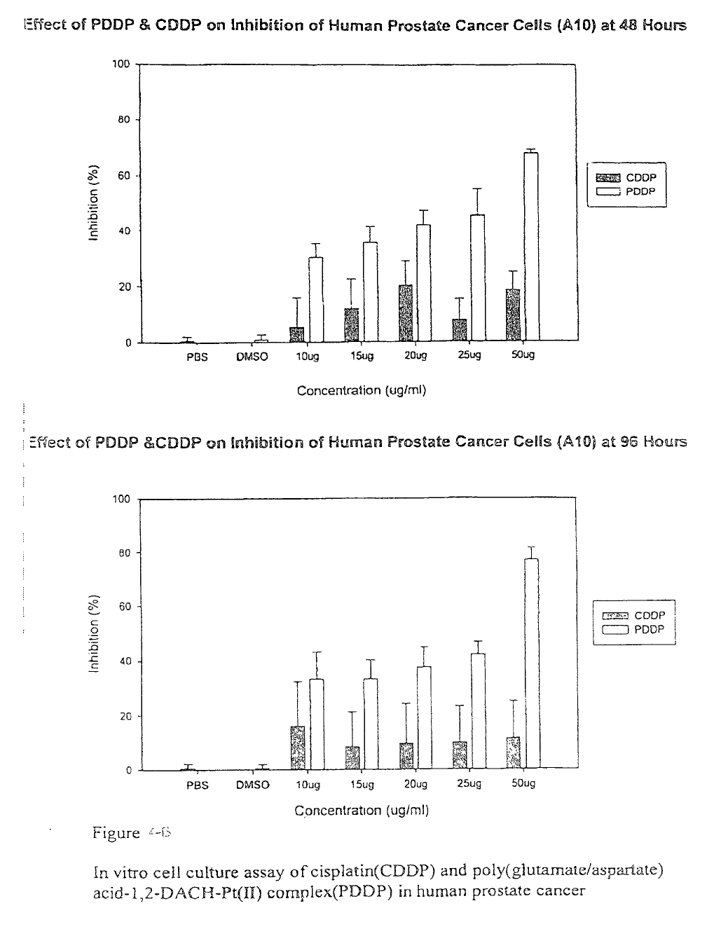

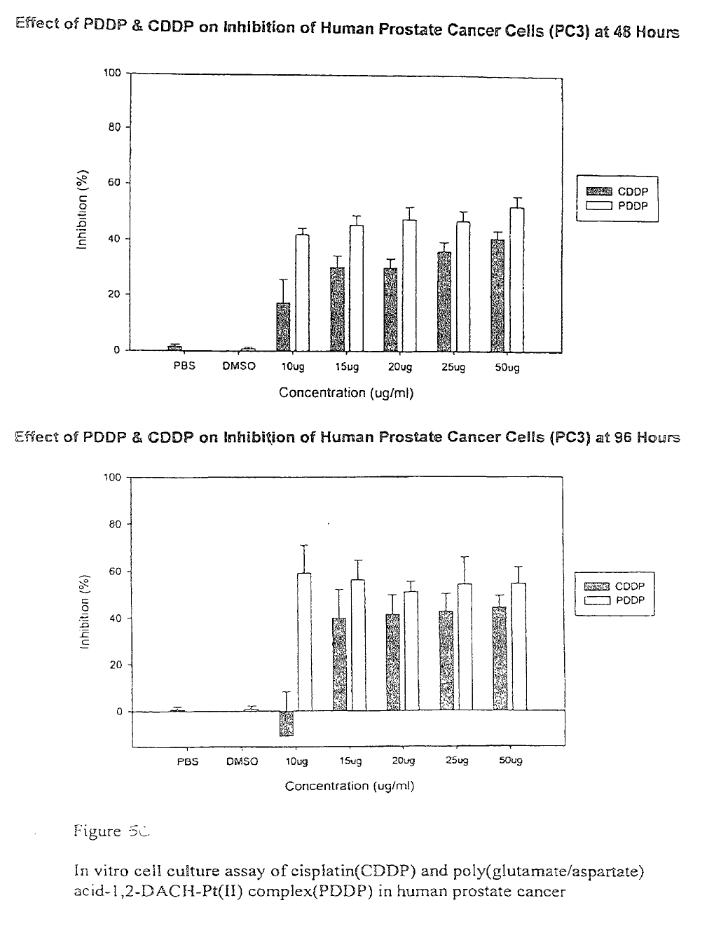

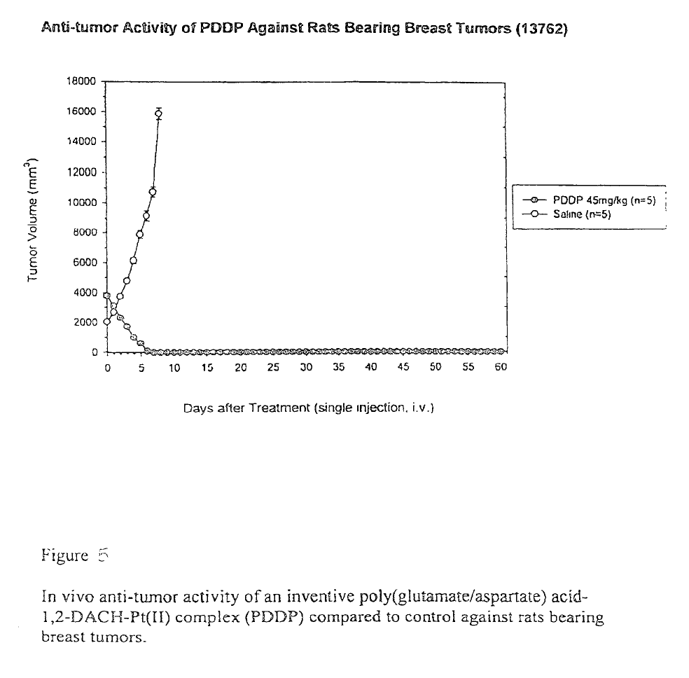

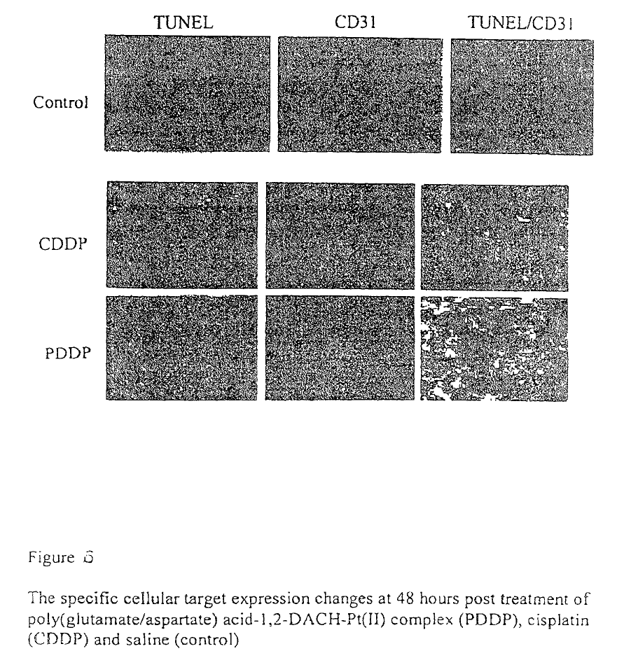

FIG. 2 provides the structures of the Pt(II) and PT(IV) complexes, and FIG. 3 provides the results of elemental analysis of platinum-poly(glutamate/aspartate) complexes Pt(II) (PDDP), Pt(IV) (PPAP), and cis-1,2-DACH—Pt SO₄(DACH).

### Example 3

In Vitro Cell Culture Assay:

To evaluate cytotoxicity of cisplatin and platinum (II) and (IV) polypeptide complex against mammary tumor cells, three human tumor cell lines were selected: PC3 (prostate); A10 (prostate); and sarcoma. All cells were cultured at 37° C. in a humidified atmosphere containing 5% CO₂ in Eagle's medium. Forty-eight hours prior to the experiment, the cells were transferred to 35 mm culture dishes at 5×10⁵ cells per dish and grown to 80% confluence. Cultured human tumor cells in 35 mm dishes were incubated with either cisplatin or platinum (II) and (IV) polypeptide complex at various concentrations. The incubation was stopped at 72 hours. Methylene tetrazolium (MTT) dye assay determined the amount of viable cells. Cellular protein content was determined by Lowry assay. The drug concentration that inhibits 50% of cell growth was then determined.

FIGS. 4A-C shows results from in vitro cell culture assays of cisplatin (CDDP) and poly(glutamate/aspartate) acid-1,2-DACH—Pt (II) complex (PDDP) in sarcoma (4A) and prostate cancer cell lines (4B and 4C).

### Example 4

Evaluation of the Conjugates in Four Tumor-Bearing Animal Models

Cisplatin is known to produce an anticancer effect against breast and ovarian tumors. Therefore, four animal models were selected: ovarian, breast and two prostate cancer models. The breast and ovarian animal models were driven from animal tumor cell lines, the prostate models were created using human cell lines xenografted in nude mice. An illustrated Polv(glutamate/aspartate)-platinum analogue (II) complex against breast tumor growth curve is shown in FIG. 5.

Breast Tumor-Bearing Animal Model:

Female Fischer 344 rats (125-175g) were inoculated with breast cancer cells (13762NF, 10⁶ cells/rat, s.c. in the hind leg). After 15-20 days and a tumor volume of 1 cm, the breast tumor-bearing rats were administered either the platinum peptide complex or cisplatin at doses of 43 mg Pt/kg (peptide platinum II) or 12 mg/kg (cisplatin). Tumor volumes and body weight were recorded daily for sixty days. Tumor volumes were measured as [length (1)×width (w)×thickness (h)]/2. Loss of body weight of 15% is considering a chemical-induced toxic effect. The results are shown in FIG. 5 and indicate that the inventive platinum peptide complexes are all effective in vivo against breast cancer.

### Example 5

Imunohistopathology of Tumor Tissue after Treatment:

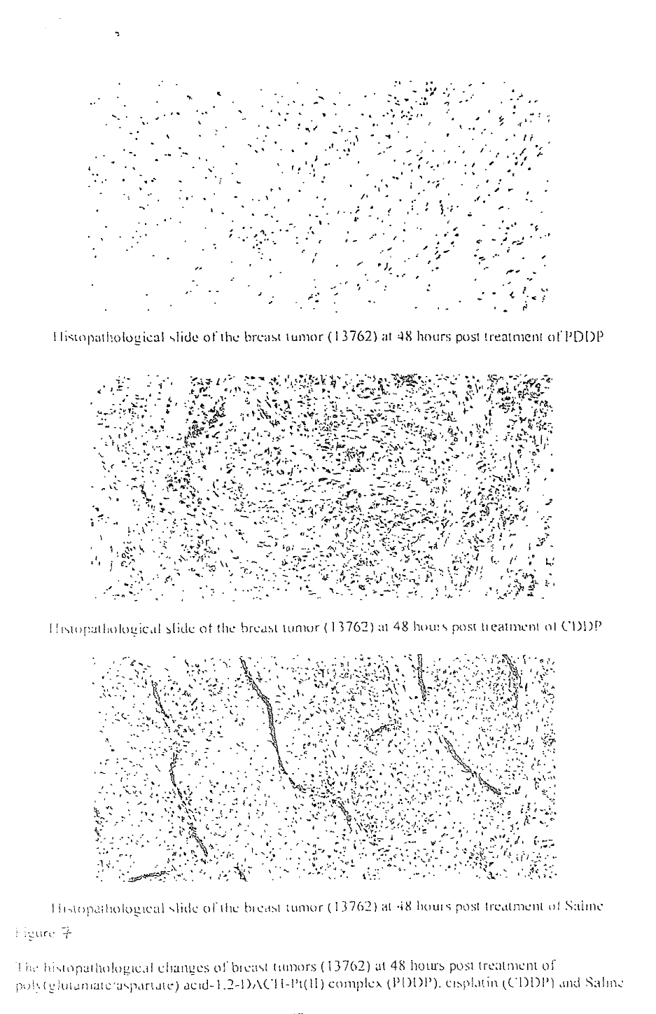

After treatment with either cisplatin or the platinum peptide complex, tumor tissues (breast) were dissected and embedded in formalin. The tumor tissue was fixed in paraffin and stained for cellular target expression. The result is shown in FIGS. 6 and 7.

FIG. 6 shows specific cellular target expression changes at 48 hours post treatment of cells with poly(glutamate/aspartate)-1,2-DACH—Pt (II) complex (PDDP), cisplatin (CDDP), and saline (Control). FIG. 7 shows histopathological changes at 48 hours post treatment of poly(glutamate/aspartate)-1,2-DACH—Pt (II) complex (PDDP), cisplatin (CDDP), and saline (Control). A marked necrosis and apoptosis were noted post-treatment.

In summary, a new polypeptide based water soluble platinum complex is developed. The solubility is increased up to 20 mg/mI. The half-life of in vitro stability in phosphate buffered saline (pH 7.4) is 18 days. The product is easily scaled up and prepared as a sterilized powder. Compared to cisplatin, insignificant toxicity was observed and much higher initial loading dose could be administered intravenously. The product produced significant effects in cancer models.

We claim:

1. A therapeutic compound comprising
a) at least one drug moiety; and
b) at least one polypeptide drug carrier moiety,
wherein the drug moiety is covalently linked to the carrier moiety, and
wherein based on the total weight of the carrier moiety, the carrier moiety comprises from about 50% to about 90% glutamic acid, and from about 10% to about 50% of at least a second amino acid selected from the group consisting of aspartic acid, alanine, asparagine, glutamine, glycine, and any combinations thereof.

2. The therapeutic compound of claim 1, wherein the drug carrier moiety has a molecular weight from about 20,000 daltons to about 50,000 daltons

3. The therapeutic compound of claim 1, wherein the second amino acid is aspartic acid.

4. The therapeutic compound of claim 1, wherein the second amino acid consists of a combination of two or more amino acids selected from the group consisting of aspartic acid, alanine, asparagine, glutamine, and glycine.

5. The therapeutic compound of claim 1, wherein the drug moiety is a therapeutic metal.

6. The therapeutic compound of claim 5, wherein the metal is selected from the group consisting of platinum, iron, gadolinium, rhenium, manganese, cobolt, indium, gallium or rhodium.

7. The therapeutic compound of claim 1, wherein the drug moiety is 1,2-diaminocyclohexane platinum (II) and 1,2-diaminocyclohexane-dichloro platinum (IV).

8. The therapeutic compound of claim 1, wherein based on the total weight of the carrier moiety, the carrier moiety comprises from about 60% to about 80% glutamic acid, and from about 20% to about 40% of the second amino acid.

9. The therapeutic compound of claim 8, wherein the second amino acid is aspartic acid.

10. The therapeutic compound of claim 8, wherein the second amino acid consists of a combination of two or more amino acids selected from the group consisting of aspartic acid, alanine, asparagine, glutamine, and glycine.

11. The therapeutic compound of claim 1, wherein based on the total weight of the carrier moiety, the carrier moiety comprises from about 70% to about 75% glutamic acid, and from about 25% to about 30% of the second amino acid.

12. The therapeutic compound of claim 11, wherein the second amino acid is aspartic acid.

13. The therapeutic compound of claim 11, wherein the second amino acid consists of a combination of two or more amino acids selected from the group consisting of aspartic acid, alanine, asparagine, glutamine, and glycine.

14. The therapeutic compound of claim 1 wherein based on the total weight of the therapeutic compound, the compound comprises from about 10% to about 60% drug moiety.

15. The therapeutic compound of claim 1, wherein based on the total weight of the therapeutic compound, the compound comprises from about 40 percent to about 90 percent carrier moiety.

16. The therapeutic compound of claim 1 wherein based on the total weight of the therapeutic compound, the compound comprises about 20 percent to about 50 percent drug moiety.

17. therapeutic compound of claim 1, wherein based on the total weight of the therapeutic compound, the compound comprises about 20 percent to about 40 percent drug moiety.

18. The therapeutic compound of claim 1, wherein the amino acids can be in L form, or D form, or a racemic mixture of L and D forms.

19. The therapeutic compound of claim 1 wherein the drug moiety is platinum (II) and platinum (IV),
wherein based on the total weight of the carrier moiety, the carrier moiety comprises about 70 percent glutamic acid and about 30 percent aspartic acid,
wherein the drug moiety is about 24 percent to about 30 percent by weight of the total weight of the therapeutic compound, and
wherein the molecular weight of the therapeutic compound is from about 26,000 to about 30,000 daltons.

20. A method for making a therapeutic compound, the method comprising the steps of:
a) covalently conjugating at least one drug moiety with at least one polypeptide drug carrier moiety to create a therapeutic compound,
wherein based on the total weight of the carrier moiety, the carrier moiety comprises from about 50% to about 90% glutamic acid, and from about 10% to about 50% of at least a second amino acid selected from the group consisting of aspartic acid, alanine, asparagine, glutamine, glycine, and any combinations thereof.

21. The method of claim 20, wherein the drug carrier moiety has a molecular weight from about 20,000 daltons to about 50,000 daltons

22. The method of claim 20, wherein the second amino acid is aspartic acid.

23. The method of claim 20, wherein the second amino acid consists of a combination of two or more amino acids selected from the group consisting of aspartic acid, alanine, asparagine, glutamine, and glycine.

24. The method of claim 20, wherein the drug moiety is a therapeutic metal.

25. The method of claim 24, wherein the metal is selected from the group consisting of platinum, iron, gadolinium, rhenium, manganese, cobolt, indium, gallium or rhodium.

26. The method of claim 20, wherein the drug moiety is 1,2-diaminocyclohexane platinum (II) and 1,2-diaminocyclohexane-dichloro platinum (IV).

27. The method of claim 20 wherein the drug moiety is platinum (II) and platinum (IV),
wherein based on the total weight of the carrier moiety, the carrier moiety comprises about 70 percent glutamic acid and about 30 percent aspartic acid,
wherein the drug moiety is about 24 percent to about 30 percent by weight of the total weight of the therapeutic compound, and
wherein the molecular weight of the therapeutic compound is from about 26,000 to about 30,000 daltons.

28. A composition comprising a therapeutic compound wherein the compound comprises
a) at least one drug moiety; and
b) at least one polypeptide drug carrier moiety,
wherein the drug moiety is covalently linked to the carrier moiety, and
wherein based on the total weight of the carrier moiety, the carrier moiety comprises from about 50% to about 90% glutamic acid, and from about 10% to about 50% of at least a second amino acid selected from the group consisting of aspartic acid, alanine, asparagine, glutamine, glycine, and any combinations thereof.

29. The composition of claim 28, wherein the drug carrier moiety has a molecular weight from about 20,000 daltons to about 50,000 daltons

30. The composition of claim 28, wherein the second amino acid is aspartic acid.

31. The composition of claim 28, wherein the second amino acid consists of a combination of two or more amino acids selected from the group consisting of aspartic acid, alanine, asparagine, glutamine, and glycine.

32. The composition of claim 28, wherein the drug moiety is a therapeutic metal.

33. The composition of claim 32, wherein the metal is selected from the group consisting of platinum, iron, gadolinium, rhenium, manganese, cobolt, indium, gallium or rhodium.

34. The composition of claim 28, wherein the drug moiety is 1,2-diaminocyclohexane platinum (II) and 1,2-diaminocyclohexane-dichloro platinum (IV).

35. The composition of claim 28 wherein the drug moiety is platinum (II) and platinum (IV),
wherein based on the total weight of the carrier moiety, the carrier moiety comprises about 70 percent glutamic acid and about 30 percent aspartic acid,
wherein the drug moiety is about 24 percent to about 30 percent by weight of the total weight of the therapeutic compound, and
wherein the molecular weight of the therapeutic compound is from about 26,000 to about 30,000 daltons.

36. A method for making a composition the method comprising the steps of:
a) combining a pharmaceutical carrier with a therapeutic compound to produce a composition, wherein the therapeutic compound comprises
a) at least one drug moiety; and
b) at least one polypeptide drug carrier moiety,
wherein the drug moiety is covalently linked to the carrier moiety, and
wherein based on the total weight of the carrier moiety, the carrier moiety comprises from about 50% to about 90% glutamic acid, and from about 10% to about 50% of at least a second amino acid selected from the group consisting of aspartic acid, alanine, asparagine, glutamine, glycine, and any combinations thereof.

37. The method of claim 36, wherein the drug carrier moiety has a molecular weight from about 20,000 daltons to about 50,000 daltons

38. The method of claim 36, wherein the second amino acid is aspartic acid.

39. The method of claim 36, wherein the second amino acid consists of a combination of two or more amino acids selected from the group consisting of aspartic acid, alanine, asparagine, glutamine, and glycine.

40. The method of claim 36, wherein the drug moiety is a therapeutic metal.

41. The method of claim 40, wherein the metal is selected from the group consisting of platinum, iron, gadolinium, rhenium, manganese, cobolt, indium, gallium or rhodium.

42. The method of claim 36, wherein the drug moiety is 1,2-diaminocyclohexane platinum (II) and 1,2-diaminocyclohexane-dichloro platinum (IV).

43. The method of claim 36 wherein the drug moiety is platinum (II) and platinum (IV),
wherein based on the total weight of the carrier moiety, the carrier moiety comprises about 70 percent glutamic acid and about 30 percent aspartic acid,
wherein the drug moiety is about 24 percent to about 30 percent by weight of the total weight of the therapeutic compound, and
wherein the molecular weight of the therapeutic compound is from about 26,000 to about 30,000 daltons.

44. The method of claim 36 wherein the composition is in a solid dosage form or a liquid dosage form.

45. The method of claim 36 wherein the composition is in a form selected from the group consisting of solids, capsules, tablets, powders, elixirs, syrups, emulsions, and suspensions.

46. A method for treating a patient afflicted with a condition, the method comprising the step of
a) administering a therapeutically effective amount of a therapeutic compound to a patient, wherein the compound comprises
a) at least one drug moiety; and
b) at least one polypeptide drug carrier moiety,
wherein the drug moiety is covalently linked to the carrier moiety, and
wherein based on the total weight of the carrier moiety, the carrier moiety comprises from about 50% to about 90% glutamic acid, and from about 10% to about 50% of at least a second amino acid selected from the group consisting of aspartic acid, alanine, asparagine, glutamine, glycine, and any combinations thereof.

47. The method of claim 46, wherein the drug carrier moiety has a molecular weight from about 20,000 daltons to about 50,000 daltons

48. The method of claim 46, wherein the second amino acid is aspartic acid.

49. The method of claim 46, wherein the second amino acid consists of a combination of two or more amino acids selected from the group consisting of aspartic acid, alanine, asparagine, glutamine, and glycine.

50. The method of claim 46, wherein the drug moiety is a therapeutic metal.

51. The method of claim 50, wherein the metal is selected from the group consisting of platinum, iron, gadolinium, rhenium, manganese, cobolt, indium, gallium or rhodium.

52. The method of claim 46, wherein the drug moiety is 1,2-diaminocyclohexane platinum (II) and 1,2-diaminocyclohexane-dichloro platinum (IV).

53. The method of claim 46 wherein the drug moiety is platinum (II) and platinum (IV),
wherein based on the total weight of the carrier moiety, the carrier moiety comprises about 70 percent glutamic acid and about 30 percent aspartic acid,
wherein the drug moiety is about 24 percent to about 30 percent by weight of the total weight of the therapeutic compound, and
wherein the molecular weight of the therapeutic compound is from about 26,000 to about 30,000 daltons.

54. The method of claim 46 wherein the step of administering comprises administering to the patient a therapeutic composition comprising the therapeutic compound,
wherein the composition may be administered orally or parenterally, and wherein the composition may be in a solid dosage form, a liquid dosage form, or any combination thereof.
